(19) United States
(12) Patent Application Publication
(10) Pub. No.: US 2025/0152528 A1
(43) Pub. Date: May 15, 2025

(54) RAPID RELIEF SPRAY

(71) Applicant: HALEON US HOLDINGS LLC, Wilmington, DE (US)

(72) Inventors: Shivangi Akash PATEL, Richmond, VA (US); Christopher Michael OHLHAVER, Richmond, VA (US); Fitsum Feleke SAHLE, Richmond, VA (US)

(21) Appl. No.: 18/835,393

(22) PCT Filed: Feb. 10, 2022
(86) PCT No.: PCT/US2022/015927

§ 371 Date: Aug. 2, 2024

(51) Int. Cl. A61K 31/167; A61K 9/08; A61K 31/137; A61K 47/10; A61K 47/12; A61K 47/14

(52) U.S. Cl. CPC A61K 31/167; A61K 9/08; A61K 31/137; A61K 47/10; A61K 47/12; A61K 47/14

(57) ABSTRACT

An aqueous composition comprising lidocaine and at least two weak acids, each having at least one carboxylic acid group with a pKa of 2 to 5, wherein the aqueous composition has a pH of 3.5 to 4.1. The aqueous composition can further comprise phenylephrine HCl.

### TECHNICAL FIELD

The invention is generally related to pharmaceutical compositions, and, more specifically, to sprayable liquid pharmaceutical compositions.

### BACKGROUND

Hemorrhoids are a common anorectal condition where veins in the anus and lower rectum become swollen. It is estimated that nearly 3 out of four adults will have hemorrhoids at some point in their life. There are two main types of hemorrhoids—internal and external. Internal hemorrhoids are usually painless unless they become thrombosed or necrotic and are often only discovered when rectal bleeding occurs during or following a bowel movement. External hemorrhoids are different from internal hemorrhoids in that they are often very painful if touched, and are commonly accompanied by itching or irritation, swelling, and bleeding in the anorectal region.

A variety of commercial products are available with various active agents that treat and/or relieve the symptoms of hemorrhoids. One example is a combination of lidocaine and phenylephrine. Lidocaine is an anesthetic that can be applied directly to a hemorrhoid to provide rapid relief through a numbing effect. Phenylephrine is a vasoconstrictor that, when applied to a hemorrhoid, can cause the hemorrhoid to shrink. Used in combination, lidocaine and phenylephrine can provide rapid relief and vasoconstriction to a user with painful hemorrhoids.

While this combination is desirable, there are surprisingly few commercial products available having this combination. One reason is that the optimal physical conditions for lidocaine are different from those of phenylephrine. Phenylephrine is stable in solutions having a pH of 4.0 or lower. In contrast, Applicants have discovered that the free-base and weak acid salt forms of lidocaine are stable in acidic solutions having a pH of at least 4.5 or higher. When phenylephrine is subjected to the higher pH solutions favorable to lidocaine, the phenylephrine begins to degrade, with the rate of degradation increasing as the pH increases. Conversely, Applicants have further discovered that when the free-base and weak acid salt forms of lidocaine are introduced into solutions having low pH conditions optimal for phenylephrine, lidocaine precipitates out of solution. Commercial products of phenylephrine and free-base or weak acid salt forms of lidocaine have thusly been plagued with undesirable short shelf lives and product instability, which is at least one reason for the lack of commercial products with this combination.

The traditional approach to the incompatibility/stability problem has been to make the hydrochloric acid (HCl) salt form of lidocaine (lidocaine HCl). The lidocaine HCl has been observed to increase the solubility of lidocaine at lower pH's, such as phenylephrine-friendly solutions having a pH of 4.0 or lower. However, the HCl salt form is problematic, because lidocaine HCl does not comply with United States Pharmacopeia National Formulary (NF) standards (hereinafter “monograph”) and the standards described in 21 C.F.R. § 346 of the US Federal Regulations pertaining to Anorectal Drug Products For Over-The-Counter Human Use (hereinafter “§ 346”). This means that lidocaine HCl and phenylephrine-containing products are severely restricted in the types of therapeutic claims permitted to be made without conducting expensive human clinical trials and submitting a new drug application to the U.S. Food and Drug Administration (“FDA”) for approval. See § 346.50 Labeling of anorectal drug products. Moreover, HCl is highly corrosive, presents manufacturing issues when used to adjust pH of a solution, especially when the manufacturing is large scale, commercial batches, and is a severe skin irritant when used in excess amounts as a pH modifier.

Accordingly, there is a need for improved spray formulations that achieve or alleviate one or more of the deficiencies found in conventional spray formulations while conforming with the monograph and § 346.

### SUMMARY

In one aspect, an aqueous composition comprises lidocaine and at least two weak acids, each having at least one carboxylic acid group with a pKa of 2 to 5, wherein the aqueous composition has a pH of 3.5 to 4.1. In some cases, the composition comprises 2 to 5 wt. % lidocaine.

In some embodiments, the composition further comprises phenylephrine HCl. The composition can comprise 0.25 wt. % phenylephrine HCl.

In some cases, each of the at least two weak acids in compositions described herein is citric acid, malic acid, acetic acid, tartaric acid, or ascorbic acid. In an embodiment, one of the at least two weak acids is citric acid. The other of the at least two weak acids can be malic acid, acetic acid, tartaric acid, or ascorbic acid in some instances.

In another embodiment, one of the at least two weak acids is malic acid. The other of the at least two weak acids can citric acid, acetic acid, tartaric acid, or ascorbic acid in some cases.

In an embodiment, the at least two weak acids in the composition are citric acid and malic acid.

The at least two weak acids can be present in a ratio of 5:3 to 1:3 based on wt. % in some cases, and in a specific instance, the composition comprises 3.6 wt. % citric acid and 1 wt. % malic acid.

In some embodiments, the aqueous composition can further comprise one or more of an antioxidant, a chelating agent, an emollient, a humectant, a preservative, a cooling agent, and a propellant. The antioxidant can comprise propyl gallate, ascorbic acid, sodium metabisulfite, potassium metabisulfite, alpha tocopherol, or any combination thereof in some cases. In some embodiments, the antioxidant comprises 0.01 to 0.1 wt. % propyl gallate.

In some instances, the chelating agent comprises disodium ethylenediaminetetraacetic acid (“EDTA”), edetic acid, or a combination of both. In some cases, the emollient comprises one or more of Aloe Barbadensis leaf juice, shea butter, vitamin E or pharmaceutically acceptable salts thereof, or any combination thereof. In some embodiments, the humectant comprises one or more of propylene glycol, glycerin, or any combination thereof. The preservative can comprise one or more of sodium benzoate, benzyl alcohol, methyl paraben, propyl paraben, or any combination thereof in some instances.

The cooling agent can comprise menthol. In some instances, menthol is present in an amount of 0.001 to 0.08 wt. %, based on a total weight of the composition.

Compositions described herein can further comprise ethanol, isopropanol, or a combination thereof in some embodiments.

In some embodiments, compositions described herein can further comprise a propellant. Exemplary propellants comprise isobutane, propane, N-butane, or any combination thereof.

In another aspect, an aqueous composition comprises 2 to 5 wt. % lidocaine; 0.25 wt. % phenylephrine HCl; and 3.5 wt. % citric acid; 1 to 5 wt. % malic acid, wherein the aqueous composition has a pH of 3.5 to 4.1. In some cases, the composition further comprises 10 to 20 wt. % glycerin. The composition further comprises 0.01 to 0.1 wt. % propyl gallate in some instances. In some embodiments, the composition further comprises 0.001 to 0.08 wt. % menthol. In some instances, the composition further comprises 5 to 10 wt. % ethanol.

In another aspect, a method of treating pain or inflammation in a mammalian subject comprising topically administering a therapeutically effective amount of a composition described herein to a mammalian subject in need thereof.

In yet another aspect, a spray device comprises a composition described herein.

### BRIEF DESCRIPTION OF THE DRAWINGS

FIG. 1 is a photographic image of lidocaine citrate-containing aqueous solutions having various pH values.

FIGS. 2a-2d are photographic images of lidocaine citrate-containing aqueous solutions having different crystallization inhibitors at different pH values.

FIG. 3 is a photographic image of lidocaine citrate-containing aqueous solutions having different alcohol cosolvents.

### DETAILED DESCRIPTION

Embodiments described herein can be understood more readily by reference to the following detailed description, examples, and figures. Elements, apparatus, and methods described herein, however, are not limited to the specific embodiments presented in the detailed description, examples, and figures. It should be recognized that the exemplary embodiments herein are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should generally be considered to include the end points 5 and 10.

It is further to be understood that the feature or features of one embodiment may generally be applied to other embodiments, even though not specifically described or illustrated in such other embodiments, unless expressly prohibited by this disclosure or the nature of the relevant embodiments. Likewise, compositions and methods described herein can include any combination of features and/or steps described herein not inconsistent with the objectives of the present disclosure. Numerous modifications and/or adaptations of the compositions and methods described herein will be readily apparent to those skilled in the art without departing from the present subject matter.

Unless expressly defined otherwise, all references to weight percentage (wt. %) refer to a weight percentage based on a total weight of the composition.

As previously discussed, for a composition with lidocaine and phenylephrine HCl to comply with the monograph and § 346, the lidocaine is required to be added to the composition in the free base form. One of the technical challenges is that the pH of the composition needs to be acidic for purposes of phenylephrine stability, such as having a pH of 4.5 or lower, or ideally 4.1 or lower. However, as was also discussed, the required free base form of lidocaine is not soluble in aqueous solutions at these pH values, and precipitates out (See Example 1). While using strong acid salt forms of lidocaine, such as lidocaine HCl, reduce or eliminate lidocaine precipitation, lidocaine HCl is not permitted under the monograph and § 346, and the use of excess HCl to adjust the pH to the proper acidic levels is highly undesirable due to the extreme discomfort of HCl on sensitive surfaces (such as on hemorrhoids). Consequently, alternative forms and/or methods are needed to produce stable compositions of lidocaine and phenylephrine that comply with the monograph and § 346.

As described in more detail in the Examples Section V herein, various approaches were explored for reducing or eliminating crystallization of lidocaine weak acid salt forms in low-pH solutions of phenylephrine. For example, the use of crystallization inhibitors, different solvent systems, and the creation of in situ ionized lidocaine weak acid salt forms/methods were all explored. However, each of these approaches were unsuccessful.

Different crystallization inhibitors were explored as a possible solution to lidocaine crystallization. As described in more detail in Example 2, the use of crystallization inhibitors failed to prevent lidocaine crystallization in low pH solutions.

Various solutions having different combinations of solvents, such as aqueous alcohol solutions, were also explored as a possible answer to the lidocaine crystallization problem. While the use of aqueous alcohol solutions was found to improve certain physical characteristics of the composition (such as improved drying time), lidocaine crystallization was still observed in low pH solutions, as described in more detail in Example 3.

In situ generation of the citrate salt form of lidocaine was investigated for increasing lidocaine solubility in low pH solutions but was ultimately unsuccessful. While lidocaine citrate was observed to increase solubility of lidocaine in mildly acidic solutions (pH=>4.6), lidocaine citrate was also observed to crystallize out in lower pH solutions (pH=<4.5). The unsuccessful use of the citrate salt version of lidocaine is detailed in Example 4 herein.

While lidocaine citrate is insoluble at lower pH solutions, there are many advantages to using lidocaine citrate as the weak acid salt form. Some of these advantages include the relatively mild acidity of citric acid, the compatibility of excess citric acid for use as a pH modifier to acidify aqueous solutions used for sensitive areas, the established safety profile of citric acid in pharmaceutical preparations, exfoliant properties, the relatively low cost of goods, and the pleasing citrus fragrance. Therefore, the use of lidocaine citrate is desirable if the problem of low-pH crystallization could be solved.

As described in more detail hereafter, it has been discovered that the use of at least two weak acids in combination increase solubility of lidocaine in low pH solutions of pH=<4.5, such as 3.5-4.1, and prevent lidocaine crystallization. Specifically, it has been found in some embodiments that the use of at least two weak acids having at least one carboxylic acid group with a pKa of 2 to 5 can increase solubility of lidocaine in low pH solutions, as detailed in Example 5 herein. In preferred embodiments, the use of citric acid in combination with a second weak acid having at least one carboxylic acid group with a pKa of 2 to 5 can increase the solubility of lidocaine in low pH solutions. Additionally, it has been discovered that an excess of citric acid can be used in combination with the second weak acid to lower the pH of the solution/composition to a pH=3.5-4.1 without adversely affecting lidocaine citrate solubility, with the additional benefit of eliminating the need for a buffer in some embodiments.

Compositions described herein, when applied to a human or mammalian skin, can in some cases eliminate or reduce one or more of the deficiencies found in conventional formulations and/or the use of strong acid salt forms of lidocaine, such as lidocaine HCl. Additionally, in preferred embodiments, the compositions can be used to provide relief to users suffering from hemorrhoids or other skin ailments.

### I. COMPOSITIONS

In an aspect, aqueous compositions described herein have a pH of 3.5 to 4.1 and comprise lidocaine and at least two weak acids, each weak acid having at least one carboxylic acid group with a pKa of 2 to 5. In preferred embodiments, the aqueous composition comprises a combination of lidocaine and phenylephrine HCl. In further embodiments, aqueous composition described herein comprise a combination of 1) lidocaine and phenylephrine HCl, and 2) citric acid and a second weak acid having at least one carboxylic acid group with a pKa of 2 to 5 can increase the solubility of lidocaine in low pH solutions. As previously discussed, lidocaine is an anesthetic that can be applied directly to a hemorrhoid, skin ulcers, insect bites, varicose veins, and minor injuries and wounds to provide rapid relief through a numbing effect. Phenylephrine is a vasoconstrictor that, when applied to a hemorrhoid, can cause the hemorrhoid to shrink slightly. Additionally, phenylephrine possesses hemostatic properties, meaning that minor bleeding can sometimes be stopped upon application to an affected area. When phenylephrine is used in combination with lidocaine, a user can experience rapid relief at affected areas, such as areas having painful hemorrhoids, insect bites, wounds, and the like.

Compositions described herein comprise therapeutically active amounts of lidocaine sufficient to provide a numbing effect, and in compositions further comprising phenylephrine, sufficient to provide hemostatic properties to a user upon application. In preferred embodiments, quantities of lidocaine and phenylephrine present in the composition comply with United States Pharmacopeia National Formulary (NF) standards (hereinafter “monograph”) and/or the standards described in 21 C.F.R. § 346 of the US Federal Regulations pertaining to Anorectal Drug Products For Over-The-Counter Human Use (hereinafter “§ 346”). In less preferred embodiments, the quantities of lidocaine and phenylephrine present in the composition can be outside the ranges specified in the monograph and/or § 346. For example, in cases where the composition complies with the monograph and/or § 346, lidocaine can be present in the composition in amounts of about 2-5 wt. %, 2.2-5 wt. %, 2.5-5 wt. %, 2.7-5 wt. %, 3-5 wt. %, 3.2-5 wt. %, 3.5-5 wt. %, 3.7-5 wt. %, 4-5 wt. %, 4.2-5 wt. %, 4.5-5 wt. %, 4.7-5 wt. %, 2-4.7 wt. %, 2-4.5 wt. %, 2-4.2 wt. %, 2-4 wt. %, 2-3.7 wt. %, 2-3.5 wt. %, 2-3.2 wt. %, 2-3 wt. %, 2-2.7 wt. %, 2-2.5 wt. %, 2-2.2 wt. %, 2.5-4.5 wt. %, 3-4 wt. %, 2 wt. %, 2.2 wt. %, 2.4 wt. %, 2.5 wt. %, 2.7 wt. %, 3 wt. %, 3.2 wt. %, 3.4 wt. %, 3.5 wt. %, 3.7 wt. %, 4 wt. %, 4.2 wt. %, 4.4 wt. %, 4.5 wt. %, 4.7 wt. %, or 5 wt. %. In instances where the composition does not comply with the monograph or § 346, lidocaine can be present in the amounts of less than 2 wt. %, such as between 0.2-1.8 wt. %, which includes at least about 1.8 wt. %, 1.6 wt. %, 1.5 wt. %, 1.4 wt. %, 1.2 wt. %, 1 wt. %, 0.8 wt. %, 0.6 wt. %, 0.5 wt. %, 0.4 wt. %, or 0.2 wt. %. In further embodiments, lidocaine can be present in the composition in amounts greater than 5 wt. %, such as between 5-10 wt. %, which includes at least about 5.5 wt. %, 6 wt. %, 6.5 wt. %, 7 wt. %, 7.5 wt. %, 8 wt. %, 8.5 wt. %, 9 wt. %, 9.5 wt. %, or 10 wt. %.

It is noted that the monograph and § 346 require lidocaine to be incorporated as the free base when the composition is being prepared. Therefore, the initial form of lidocaine is the free base when the composition is being prepared in embodiments complying with these sections. However, in other cases, when outside the monograph and/or § 346, lidocaine can be in a salt form described herein prior to addition to the composition. The lidocaine amounts described herein are for the lidocaine free-base form initially added to the composition.

When the composition complies with the monograph and/or § 346, phenylephrine is required by these guidelines to be in the hydrochloric acid (HCl) salt form when the composition is being prepared. Consequently, in embodiments where the composition complies with the monograph and/or § 346, phenylephrine is used as phenylephrine HCl to prepare the composition. Similar to lidocaine, in other cases, when outside the monograph and/or § 346, phenylephrine can be in a free base form or in an alternative salt form prior to addition to the composition.

Additionally, in embodiments where the composition complies with the monograph and/or § 346 for combinations of lidocaine and phenylephrine HCl, phenylephrine HCl can be present in the composition in an amount of about 0.25 wt. %. In other embodiments, compositions can have amounts of phenylephrine HCl outside the amount specified in the monograph and/or § 346, such as lower amounts of about 0.01-0.22 wt. %, including 0.01 wt. %, 0.05 wt. %, 0.08 wt. %, 0.1 wt. %, 0.15 wt. %, 0.18 wt. %, 0.2 wt. %, or 0.22 wt. %; or higher amounts of about 0.27-3 wt. %, including 0.35 wt. %, 0.5 wt. %, 0.65 wt. %, 0.75 wt. %, 0.85 wt. %, 1 wt. %, 1.2 wt. %, 1.5 wt. %, 1.7 wt. %, 2 wt. %, 2.2 wt. %, 2.5 wt. %, 2.7 wt. %, or 3 wt. %.

As previously discussed, the free-base form of lidocaine is insoluble in aqueous solutions, and weak acid salts of lidocaine have been observed to precipitate out of aqueous solutions when excess weak acid is added to lower pH. It has been found that the use of two weak acids in combination with lidocaine can increase the solubility of lidocaine at lower pH's. While not intending to be bound by theory, it is believed that the use of certain single weak acids, such as citric acid, causes lidocaine precipitation at lower pH's due to the common ion effect, where, as excess citric acid precipitates out of solution at lower pH's, lidocaine citrate is pulled out of solution at the same time through co-crystallization. It is believed that the addition of a second weak acid maintains solubility of lidocaine, resulting in lidocaine remaining solubilized in the solution. Additionally, the second weak acid is believed to form a salt with lidocaine, reducing the concentration of lidocaine citrate in the solution, which in turn also increases lidocaine solubility. Compositions described herein have a combination of at least two weak acids. In some embodiments, each weak acid has at least one carboxylic acid group with a pKa of 2 to 5, 2.5 to 5, 3 to 5, 3.5 to 5, 4 to 5 4.5 to 5, 2 to 4.5, 2 to 4, 2 to 3.5, 2 to 3, 2 to 2.5, 2.5-4.5, or 3 to 4. Exemplary weak acids include citric acid, malic acid, acetic acid, tartaric acid, or ascorbic acid. In some embodiments, the weak acid comprises citric acid in combination with malic acid, acetic acid, tartaric acid, or ascorbic acid. In another embodiment, the combination of at least two weak acids includes a combination of citric acid and malic acid.

The ratio of the two weak acids can be any ratio not inconsistent with the objectives of this disclosure. In some embodiments, the two weak acids are present in a ratio of 5:3 to 1:3, 4.8:3 to 1:3, 4:5 to 1:3, 4.6:3 to 1:3, 4.4:3 to 1:3, 4.2:3 to 1:3, 4:3 to 1:3, 3.8:3 to 1:3, 3.6:3 to 1:3, 3.4:3 to 1:3, 3.2:3 to 1:3, 3:3 to 1:3, 2.8:3 to 1:3, 2.6:3 to 1:3, 2.4:3 to 1:3, 2.2:3 to 1:3, 2:3 to 1:3, 1.8:3 to 1:3, 1.6:3 to 1:3, 1.4:3 to 1:3, 1.2:2 to 1:3, 5:3 to 1.2:3, 5:3 to 1.4:3, 5:3 to 1.6:3, 5:3 to 1.8:3, 5:3 to 2:3, 5:3 to 2.2:3, 5:3 to 2.4:3, 5:3 to 2.6:3, 5:3 to 2.8:3, 5:3 to 3:3, 5:3 to 3.2:3, 5:3 to 3.4:3, 5:3 to 3.6:3, 5:3 to 3.8:3, 5:3 to 4:3, 5:3 to 4.2:3, 5:3 to 4.4:3, 5:3 to 4.6:3, 5:3 to 4.8:3, 5:3, 4.8:3, 4.6:3, 4.4:3, 4.2:3, 4:3, 3.8:3, 3.6:3, 3.4:3, 3.2:3, 3:3, 2.8:3, 2.6:3, 2.4:3, 2.2:3, 2:3, 1.8:3, 1.6:3, 1.4:3, 1.2:3, 1:3, 1:3.2, 1:3.4, 1:3.6, 1:3.8, 1:4, 1:4.2, 1:4.4, 1:4.6, 1:4.8, or 1:5, where the ration is based on wt. % of the composition.

In some embodiments, the aqueous composition comprises between 2.6-4.0 wt. %, 2.6 wt. %, 2.8 wt. %, 3.0 wt. %, 3.2 wt. %, 3.4 wt. %, 3.6 wt. %, 3.8 wt. %, or 4.0 wt. % of citric acid in combination with a weak acid having at least one carboxylic acid group with a pKa of 2 to 5, 2.5 to 5, 3 to 5, 3.5 to 5, 4 to 5 4.5 to 5, 2 to 4.5, 2 to 4, 2 to 3.5, 2 to 3, 2 to 2.5, 2.5-4.5, or 3 to 4. In one embodiment, the aqueous composition comprises 1 wt. % malic acid and 3.6 wt. % citric acid. In another instance, the aqueous composition comprises 2 wt. % malic acid and 3.6 wt. % citric acid. In another case, the aqueous composition comprises 5 wt. % malic acid and 3.6 wt. % citric acid.

In some embodiments, the aqueous composition has a pH of 3.5 to 4.1, 3.5 to 4.0, 3.5 to 3.9, 3.5 to 3.8, 3.5 to 3.7, 3.5 to 3.6, 3.6 to 4.1, 3.7 to 4.1, 3.8 to 4.1, 3.9 to 4.1, 3.6 to 4.0, 3.7 to 3.9, 3.5, 3.6, 3.7, 3.8, 3.9, 4.0, or 4.1. As previously discussed, the aqueous composition pH can be adjusted through the addition of one of the at least two weak acids without the need for additional buffer components. For example, an amount of citric acid in excess of the amount needed to form lidocaine citrate in situ can be added to the aqueous composition to adjust the pH to the desired level. Similarly other weak acids described herein can be used in excess to adjust the pH in some cases.

Notably, it has been observed that some weak acids can be used by themselves to form lidocaine salts that remain soluble in aqueous solutions at lower pH's without the addition of a second weak acid, such as malic acid. Acetic acid-based salt (lidocaine acetate) is believed to also remain soluble in aqueous solutions at lower pH's. However, these single salt forms are problematic in many ways. For example, one of the benefits of using citric acid as the salt form of lidocaine is that the pH can be adjusted to be between 3.5 to 4.1 using an excess of citric acid without any issues with skin irritation or other undesirable side effects. A further advantage of citric acid is that, since it functions as both the salt form of lidocaine and a pH modifier, compositions described herein can be buffer-free, reducing composition complexity and cost.

In contrast, other alpha-hydroxy acids such as malic acid have very undesirable side effects when used in concentrations of >5 wt. % in aqueous solutions. At levels below 5 wt. % (such as in embodiments described herein), malic acid is a beneficial skin exfoliant, but when used above 5 wt. %, malic acid can be a severe skin irritant—a problematic side effect when treating a hemorrhoid or skin laceration. To adjust the pH of compositions described herein to be between 3.5 to 4.1, greater than 5 wt. % of malic acid is used, exceeding the beneficial range and entering into the undesirable skin irritant range. Thus, low levels of malic acid (<5 wt. %) can be used in combination with citric acid to impart the desired lidocaine citrate solubility properties and to receive the beneficial exfoliating properties of malic acid, and excess citric acid can be used to obtain the desired lower pH without the need for a pH buffer. See Example 4 for more details on the use of malic acid as a single salt form of lidocaine in low pH aqueous solutions.

While not intending to be bound by theory, acetic acid as a single weak acid salt form of lidocaine is similarly undesirable. Being a liquid at room temperature, when excess acetic acid used as a pH modifier, it imparts a strong, unpleasant vinegar smell to the composition, and the excess acetic acid may also be a skin irritant in some cases. Consequently, while both malic acid and acetic acid can address lidocaine solubility at lower pH's in some cases as the single weak acid salt form of lidocaine, neither can be used in excess as a pH modifier without unpleasant side effects, with both necessitating the use of a buffer.

In some embodiments, compositions described herein can further comprise one or more excipients. Exemplary excipients can comprise an antioxidant, a chelating agent, an emollient, a humectant, a preservative, a cooling agent, a propellant or any combination thereof.

As previously discussed, phenylephrine HCl is unstable in solutions with a pH of greater than approximately 4. In some cases, inclusion of one or more antioxidants in the composition can assist in the reduction in the rate or elimination of phenylephrine HCl degradation. Exemplary antioxidants comprise propyl gallate, ascorbic acid, sodium metabisulfite, potassium metabisulfite, alpha tocopherol, or any combination thereof. In one embodiment, the antioxidant is propyl gallate.

Each of the one or more antioxidants can be present in any amount not inconsistent with the objectives of this disclosure. For example, when the antioxidant is propyl gallate, propyl gallate can be present between 0.01 to 0.1 wt. %, 0.02 to 0.1 wt. %, 0.03 to 0.1 wt. %, 0.04 to 0.1 wt. %, 0.05 to 0.1 wt. %, 0.06 to 0.1 wt. %, 0.07 to 0.1 wt. %, 0.08 to 0.1 wt. %, 0.09 to 0.1 wt. %, 0.01 to 0.09 wt. %, 0.01 to 0.08 wt. %, 0.01 to 0.07 wt. %, 0.01 to 0.06 wt. %, 0.01 to 0.05 wt. %, 0.01 to 0.04 wt. %, 0.01 to 0.03 wt. %, 0.01 to 0.02 wt. %, 0.02 to 0.08 wt. %, 0.03 to 0.07 wt. %, 0.04 to 0.06 wt. %, 0.01 wt. %, 0.02 wt. %, 0.03 wt. %, 0.04 wt. %, 0.05 wt. %, 0.06 wt. %, 0.07 wt. %, 0.08 wt. %, 0.09 wt. %, or 0.1 wt. %. The skilled artisan would appreciate that other antioxidants will have different ranges of acceptable amounts based their unique physical and chemical properties.

Any chelating agent in any quantity not inconsistent with the objectives of this disclosure can be used. Exemplary chelating agents include disodium ethylenediaminetetraacetic acid (EDTA), edetic acid, or a combination of both. When present in the composition, chelating agents can be present in amounts of 0.05-0.3 wt. %, 0.05-0.28 wt. %, 0.05-0.25 wt. %, 0.05-0.2 wt. %, 0.05-0.18 wt. %, 0.05-0.15 wt. %, 0.05-0.12 wt. %, 0.05-0.1 wt. %, 0.05-0.08 wt. %, 0.08-0.3 wt. %, 0.1-0.3 wt. %, 0.12-0.3 wt. %, 0.15-0.3 wt. %, 0.18-0.3 wt. %, 0.2-0.3 wt. %, 0.22-0.3 wt. %, 0.25-0.3 wt. %, 0.28-0.3 wt. %, up to 0.05 wt. %, up to 0.07 wt. %, up to 0.1 wt. %, 0.05 wt. %, 0.07 wt. %, 0.1 wt. %, 0.12 wt. %, 0.15 wt. %, 0.18 wt. %, 0.2 wt. %, 0.22 wt. %, 0.25 wt. %, 0.28 wt. %, or 0.3 wt. %.

Emollients described herein can be any emollient in any quantity not inconsistent with the objectives of this disclosure. Typical emollients act as moisturizers to treat, prevent or alleviate rough, dry, or itchy skin and minor skin irritations. Exemplary emollients comprise Aloe Barbadensis leaf juice, shea butter, vitamin E and vitamin E pharmaceutically acceptable salts, or any combination thereof. Emollients can be present in the composition in quantities sufficient to provide a moisturizing effect on the skin of a user. For example, an emollient can be present in the composition in amounts up to 0.1 wt. %, up to 0.5 wt. %, up to 0.8 wt. %, up to 1 wt. %, up to 1.2 wt. %, up to 1.5 wt. %, up to 1.8 wt. %, up to 2 wt. %, or up to 3 wt. %. In a preferred embodiment, the emollient is 0.1 wt. % Aloe Barbadensis leafjuice.

In some embodiments, humectants are present in the composition. While not intending to be bound by theory, humectants are believed to form a protective barrier layer over hemorrhoids that slows drying of the tissues, reduces itching sensations and increases permeability and absorption of active pharmaceutical ingredients present in the composition, such as, for example, lidocaine and phenylephrine.

Any humectant not inconsistent with the objectives of this disclosure can be used, including propylene glycol, sorbitol, glycerin, or any combination thereof. The humectant can be present in the composition in any amount not inconsistent with the objectives of this disclosure. As can be appreciated by the skilled artisan, the humectant can be present in higher amounts, such as glycerin previously described herein, with the amount of humectant being dependent on the particular humectant's physical properties and potency.

In some embodiments, compositions described herein can comprise propylene glycol as a humectant. Propylene glycol can be present in an amount of 0.5 to 5 wt. %, 1 to 5 wt. %, 1.5 to 5 wt. %, 2 to 5 wt. %, 2.5 to 5 wt. %, 3 to 5 wt. %, 3.5 to 5 wt. %, 4 to 5 wt. %, 4.5 to 5 wt. %, 0.5 to 4.5 wt. %, 0.5 to 4 wt. %, 0.5 to 3.5 wt. %, 0.5 to 3 wt. %, 0.5 to 2.5 wt. %, 0.5 to 2 wt. %, 0.5 to 1.5 wt. %, 0.5 to 1 wt. %, 1 to 4.5 wt. %, 1.5 to 4 wt. %, 2 to 3.5 wt. %, 2.5-3 wt. %, 0.5 wt. %, 1 wt. %, 1.5 wt. %, 2 wt. %, 2.5 wt. %, 3 wt. %, 3.5 wt. %, 4 wt. %, 4.5 wt. %, or 5 wt. %. In some instances, composition described herein can comprise glycerin as a humectant. Glycerin can be present in the composition in amounts of 10-45 wt. %, 10-40 wt. %, 10-35 wt. %, 10-30 wt. %, 10-25 wt. %, 10-20 wt. %, 10-15 wt. %, 15-45 wt. %, 20-45 wt. %, 25-45 wt. %, 30-45 wt. %, 35-45 wt. %, 40-45 wt. %, 10 wt. %, 15 wt. %, 20 wt. %, 25 wt. %, 30 wt. %, 35 wt. %, 40 wt. %, or 45 wt. %.

Compositions described herein can comprise a combination of at least two humectants. For example, in some instances, the composition comprises a combination of propylene glycol and glycerin, each in the amount previously described herein, such as 0.5 to 5 wt. % propylene glycol and 10-45 wt. % glycerin.

In some instances, a preservative is present in the composition. The preservative can be any preservative not inconsistent with the objectives of this disclosure. Exemplary preservatives include sodium benzoate, benzyl alcohol, methyl paraben, propyl paraben, or any combination thereof. The preservative can be present in the composition in amounts between 0.1 to 2 wt. %, 0.2 to 2 wt. %, 0.3 to 2 wt. %, 0.4 to 2 wt. %, 0.5 to 2 wt. %, 0.6 to 2 wt. %, 0.7 to 2 wt. %, 0.8 to 2 wt. %, 0.9 to 2 wt. %, 1 to 2 wt. % 1.2 to 2 wt. %, 1.4 to 2 wt. %, 1.6 to 2 wt. %, 1.8 to 2 wt. %, 0.1 to 1.8 wt. %, 0.1 to 1.6 wt. %, 0.1 to 1.4 wt. %, 0.1 to 1.2 wt. %, 0.1 to 1 wt. %, 0.1 to 0.9 wt. %, 0.1 to 0.8 wt. %, 0.1 to 0.7 wt. %, 0.1 to 0.6 wt. %, 0.1 to 0.5 wt. %, 0.1 to 0.4 wt. %, 0.1 to 0.3 wt. %, 0.1 to 0.2 wt. %, 0.2 to 0.8 wt. %, 0.3 to 0.7 wt. %, 0.4 to 0.6 wt. %, 2 wt. %, 1.8 wt. %, 1.6 wt. %, 1.4 wt. %, 1.2 wt. %, 1 wt. %, 0.9 wt. %, 0.8 wt. %, 0.7 wt. %, 0.6 wt. %, 0.5 wt. %, 0.4 wt. %, 0.3 wt. %, 0.2 wt. %, or 0.1 wt. %.

The two or more weak acids described herein can further server as pH modifiers in the composition. As previously discussed, the two or more weak acids used in the composition bring the pH of the composition to between about 3.5 to 4.1. Thus, one of the advantages of compositions herein is that the composition can be buffer-free. For example, as described in more detail in the Examples, citric acid can be used to both form the citrate salt of lidocaine and as a pH modifier to lower the pH of the composition without the need for a buffer.

Compositions described herein can optionally further include one or more cooling agents, such as camphor, menthol, juniper tar, other known cooling agents, or combinations thereof. The quantity of the additional active pharmaceutical ingredient present in the composition is specific to the physical properties of that ingredient. Thus, when present, the amount of additional active pharmaceutical ingredient can be any therapeutically effective amount not inconsistent with the objectives of this disclosure. In embodiments of compositions having camphor or menthol as a cooling agent (and not as an active ingredient), the composition comprises between 0.01 to 0.09 wt. %, 0.02 to 0.09 wt. %, 0.03 to 0.09 wt. %, 0.04 to 0.09 wt. %, 0.05 to 0.09 wt. %, 0.06 to 0.09 wt. %, 0.07 to 0.09 wt. %, 0.08 to 0.09 wt. %, 0.01 to 0.08 wt. %, 0.01 to 0.07 wt. %, 0.01 to 0.06 wt. %, 0.01 to 0.05 wt. %, 0.01 to 0.04 wt. %, 0.01 to 0.03 wt. %, 0.01 to 0.02 wt. %, 0.02 to 0.08 wt. %, 0.03 to 0.07 wt. %, 0.04 to 0.06 wt. %, 0.01 wt. %, 0.02 wt. %, 0.03 wt. %, 0.04 wt. %, 0.05 wt. %, 0.06 wt. %, 0.07 wt. %, 0.08 wt. %, or 0.09 wt. %. In the instances of compositions optionally comprising juniper tar, juniper tar can be present between 0.1 to 0.9 wt. %, 0.2 to 0.9 wt. %, 0.3 to 0.9 wt. %, 0.4 to 0.9 wt. %, 0.5 to 0.9 wt. %, 0.6 to 0.9 wt. %, 0.7 to 0.9 wt. %, 0.2 to 0.9 wt. %, 0.3 to 0.9 wt. %, 0.4 to 0.9 wt. %, 0.5 to 0.9 wt. %, 0.6 to 0.9 wt. %, 0.7 to 0.9 wt. %, 0.8 to 0.9 wt. %, 0.2 to 0.8 wt. %, 0.3 to 0.7 wt. %, 0.4 to 0.6 wt. %, 0.1 wt. %, 0.2 wt. %, 0.3 wt. %, 0.4 wt. %, 0.5 wt. %, 0.6 wt. %, 0.7 wt. %, 0.8 wt. % or 0.9 wt. %.

Compositions described herein can further comprise water. Water can be present as the balance remaining in the wt. % of the composition. For example, water can be present in amounts ranging from 30-80 wt. %, 35-80 wt. %, 40-80 wt. %, 45-80 wt. %, 50-80 wt. %, 55-80 wt. %, 60-80 wt. %, 65-80 wt. %, 70-80 wt. %, 30-75 wt. %, 35-70 wt. %, 30-65 wt. %, 30-60 wt. %, 30-55 wt. %, 30-50 wt. %, 30-45 wt. %, 30-40 wt. %, 30-35 wt. %, 35-65 wt. %, 40-60 wt. %, 45-55 wt. %, 30 wt. %, 32 wt. %, 35 wt. %, 37 wt. %, 40 wt. %, 42 wt. %, 45 wt. %, 47 wt. %, 50 wt. %, 52 wt. %, 55 wt. %, 57 wt. %, 60 wt. %, 62 wt. %, 64 wt. %, 65 wt. %, 66 wt. %, 67 wt. %, 70 wt. %, 72 wt. %, 75 wt. %, 77 wt. % or 80 wt. %.

In cases where compositions described herein are to be applied as spray formulations, the composition can comprise a propellant. Any propellant that is not inconsistent with the objectives of this disclosure can be used. For example, in some instances, a propellant can include isobutane, propane, N-butane, or any combination thereof. The propellant can be present in the composition in any amount not inconsistent with the objectives of this disclosure. For example, the propellant can be present in an amount of 5-30 wt. %, such as 5 wt. %, 10 wt. %, 15 wt. %, 20 wt. %, 25 wt. %, 30 wt. %, or any integer or fraction therebetween.

### II. METHODS

In another aspect, compositions described in Section I herein can generally be prepared by combining lidocaine, at least two of the weak acids, and water. In some embodiments, compositions can be prepared by combining the at least two of the weak acids and water, adding the lidocaine to the resulting acidic solution, and additionally combining one or more of an antioxidant, a chelating agent, an emollient, a humectant, a preservative, a cooling agent, and a propellant, or any combinations thereof described in Section I herein.

In some embodiments, compositions described in Section I herein can generally be prepared by combining lidocaine, phenylephrine HCl, at least two of the weak acids, and water. In some embodiments, compositions can be prepared by combining lidocaine, phenylephrine HCl, at least two of the weak acids, and water, and additionally combining one or more of an antioxidant, a chelating agent, an emollient, a humectant, a preservative, a cooling agent, and a propellant, or any combinations thereof described in Section I herein.

In some cases, lidocaine free base and at least two weak acids can be combined in a solvent comprising additional excipients, such as the one or more of an antioxidant, a chelating agent, an emollient, a humectant, a preservative, a cooling agent, to form the lidocaine citrate salt in situ and to adjust the pH of the resulting solution to be between 3.5-4.1 prior to addition of phenylephrine HCl. This sequential methodology can, in some embodiments, maintain phenylephrine HCl stability upon addition to the composition because the pH is in the desired range prior to the addition.

In another embodiment, the at least two weak acids and excipients (such as the one or more of an antioxidant, a chelating agent, an emollient, a humectant, a preservative, a cooling agent) are firstly combined in a solvent. This can be through sequential or simultaneous addition of the weak acids and excipients directly into the same solvent in some cases. In other cases, each individual weak acid and/or excipient or different combinations of the weak acids and/or excipients can be separately dissolved into solvents to form different pre-mixtures, and these separate pre-mixtures can then be combined to form a single solution. Lidocaine free-base can then be added to the single solution, either in powdered form or as a lidocaine-containing solution. Phenylephrine HCl can optionally be added at the same time as the weak acids and/or excipients, combined in a solved to form a pre-mixture and added to form the single solution, added as a powder or solution at the same time as the lidocaine free-base is added, or after lidocaine free-base addition.

### III. METHODS OF TREATMENT

In yet another aspect, a method of treating pain or inflammation in a mammalian subject comprising topically administering a therapeutically effective amount of any composition described in Section I herein to a mammalian subject in need thereof.

In some embodiments, a method of treating pain or inflammation exhibited by hemorrhoids, eczema, skin ulcers, insect bites, varicose veins, acne, and minor injuries and wounds comprises topically administering a therapeutically effective amount of any composition described in Section I herein to a mammalian subject in need thereof.

In some cases, a method of reducing swelling and irritation of hemorrhoids, eczema, skin ulcers, insect bites, varicose veins, acne, and minor injuries and wounds comprises topically administering a therapeutically effective amount of any composition described in Section I herein to a mammalian subject in need thereof.

In some cases, a mammalian subject is a human subject. In other cases, a mammalian subject is an animal subject.

### IV. EMBODIMENTS

The skilled artisan would understand that modifications and variations of the compositions described herein can be made within the scope of the invention. The following exemplary embodiments illustrate some, but not all variations of the invention.

Embodiment 1. An aqueous composition comprising:
lidocaine;
phenylephrine HCl; and
3.6 wt. % citric acid;
1 to 5 wt. % malic acid,
wherein the aqueous composition has a pH of 3.5 to 4.1.

Embodiment 2. The aqueous composition of Embodiment 1, comprising 2 to 5 wt. % lidocaine.

Embodiment 3. The aqueous composition of Embodiments 1 or 2, comprising 0.25 wt. % phenylephrine HCl.

Embodiment 4. An aqueous composition comprising:
2 to 5 wt. % lidocaine;
0.25 wt. % phenylephrine HCl; and
citric acid;
malic acid,
wherein the aqueous composition has a pH of 3.5 to 4.1.

Embodiment 5. The aqueous composition of Embodiment 4, wherein a ratio of malic acid to citric acid is between 5:3 to 1:3.

Embodiment 6. The aqueous composition of Embodiments 4 or 5, comprising 3.6 wt. % citric acid.

Embodiment 7. The aqueous composition of any of Embodiments 4 to 6, comprising 1 to 5 wt. % malic acid.

Embodiment 8. An aqueous composition comprising:
2 to 5 wt. % lidocaine;
0.25 wt. % phenylephrine HCl; and
at least two weak acids selected from a group consisting essentially of citric acid, malic acid, acetic acid, tartaric acid, and ascorbic acid,
wherein the aqueous composition has a pH of 3.5 to 4.1.

Embodiment 9. An aqueous composition comprising:
lidocaine;
phenylephrine HCl; and
at least two weak acids selected from a group consisting essentially of citric acid, malic acid, acetic acid, tartaric acid, and ascorbic acid,
wherein the aqueous composition has a pH of 3.5 to 4.1.

Embodiment 10. The aqueous composition of Embodiment 8, wherein a ratio of the two weak acids is between 5:3 to 1:3.

Embodiment 11. An aqueous composition comprising:
lidocaine;
phenylephrine HCl; and
citric acid in combination with at least one weak acid selected from a group consisting essentially of malic acid, acetic acid, tartaric acid, and ascorbic acid,
wherein the aqueous composition has a pH of 3.5 to 4.1.

Embodiment 12. An aqueous composition comprising:
lidocaine;
phenylephrine HCl; and
citric acid in combination with at least one weak acid having at least one carboxylic acid group with a pKa of 2 to 5,
wherein the aqueous composition has a pH of 3.5 to 4.1.

Embodiment 13. The aqueous composition of Embodiments 11 or 12, comprising 2 to 5 wt. % lidocaine.

Embodiment 14. The aqueous composition of any of Embodiments 11-13, comprising 0.25 wt. % phenylephrine HCl.

Embodiment 15. The aqueous composition of any of Embodiments 11-14, comprising 3.6 wt. % citric acid.

Embodiment 16. The aqueous composition of any of the preceding Embodiments further comprising one or more of an antioxidant, a chelating agent, an emollient, a humectant, a preservative, a cooling agent, and a propellant.

Embodiment 17. The aqueous composition of Embodiment 16, wherein the antioxidant comprises propyl gallate, ascorbic acid, sodium metabisulfite, potassium metabisulfite, alpha tocopherol, or any combination thereof.

Embodiment 18. The aqueous composition of Embodiment 17, wherein the antioxidant comprises 0.01 to 0.1 wt. % propyl gallate.

Embodiment 19. The aqueous composition of any of Embodiments 16-18, wherein the chelating agent comprises disodium ethylenediaminetetraacetic acid (“EDTA”), edetic acid, or a combination of both.

Embodiment 20. The aqueous composition of any of Embodiments 16-19, wherein the emollient comprises one or more of Aloe Barbadensis leaf juice, shea butter, vitamin E or pharmaceutically acceptable salts thereof, or any combination thereof.

Embodiment 21. The aqueous composition of any of Embodiments 16-20, wherein the humectant comprises one or more of propylene glycol, glycerin, or any combination thereof.

Embodiment 22. The aqueous composition of Embodiment 21, comprising 10 to 20 wt. % glycerin.

Embodiment 23. The aqueous composition of any of Embodiments 16-22, wherein the preservative comprises one or more of sodium benzoate, benzyl alcohol, methyl paraben, propyl paraben, or any combination thereof.

Embodiment 24. The aqueous composition of any of Embodiments 16-23, wherein the cooling agent comprises menthol.

Embodiment 25. The aqueous composition of any of Embodiments 16-24, comprising 0.001 to 0.08 wt. % menthol.

Embodiment 26. The aqueous composition of any of the preceding Embodiments, further comprising ethanol, isopropanol, or a combination thereof.

Embodiment 27. The aqueous composition of any of Embodiments 16-26, comprising 5-15 wt. % ethanol.

Embodiment 28. The aqueous composition of any of Embodiments 16-27, comprising a propellant selected from a group consisting of isobutane, propane, N-butane, and any combination thereof.

Embodiment 29. An aqueous composition comprising lidocaine and at least two weak acids, each having at least one carboxylic acid group with a pKa of 2 to 5, wherein the aqueous composition has a pH of 3.5 to 4.1.

Embodiment 30. The aqueous composition of Embodiment 29, comprising 2 to 5 wt. % lidocaine.

Embodiment 31. The aqueous composition of Embodiments 29 or 30, further comprising phenylephrine HCl.

Embodiment 32. The aqueous composition of any of Embodiments 29-31, comprising 0.25 wt. % phenylephrine HCl.

Embodiment 33. The aqueous composition of any of Embodiments 29-32, wherein each of the at least two weak acids is one of citric acid, malic acid, acetic acid, tartaric acid, or ascorbic acid.

Embodiment 34. The aqueous composition of any of Embodiments 29-33, wherein one of the at least two weak acids is citric acid.

Embodiment 35. The aqueous composition of Embodiment 34, wherein the other of the at least two weak acids is malic acid, acetic acid, tartaric acid, or ascorbic acid.

Embodiment 36. The aqueous composition of any of Embodiments 29-34, wherein one of the at least two weak acids is malic acid.

Embodiment 37. The aqueous composition of Embodiment 36, wherein the other of the at least two weak acids is citric acid, acetic acid, tartaric acid, or ascorbic acid.

Embodiment 38. The aqueous composition of any of Embodiments 29-34, wherein the at least two weak acids are citric acid and malic acid.

Embodiment 39. The aqueous composition of any of Embodiments 29-34, wherein the at least two weak acids are present in a ratio of 5:3 to 1:3 based on wt. %.

Embodiment 40. The aqueous composition of Embodiment 38 comprising 3.6 wt. % citric acid and 1 wt. % malic acid.

Embodiment 41. The aqueous composition of any of Embodiments 29-40 further comprising one or more of an antioxidant, a chelating agent, an emollient, a humectant, a preservative, a cooling agent, and a propellant.

Embodiment 42. The aqueous composition of Embodiment 41, wherein the antioxidant comprises propyl gallate, ascorbic acid, sodium metabisulfite, potassium metabisulfite, alpha tocopherol, or any combination thereof.

Embodiment 43. The aqueous composition of Embodiment 41, wherein the antioxidant comprises 0.01 to 0.1 wt. % propyl gallate.

Embodiment 44. The aqueous composition of Embodiment 41, wherein the chelating agent comprises disodium ethylenediaminetetraacetic acid (“EDTA”), edetic acid, or a combination of both.

Embodiment 45. The aqueous composition of Embodiment 41, wherein the emollient comprises one or more of Aloe Barbadensis leaf juice, shea butter, vitamin E or pharmaceutically acceptable salts thereof, or any combination thereof.

Embodiment 46. The aqueous composition of Embodiment 41, wherein the humectant comprises one or more of propylene glycol, glycerin, or any combination thereof.

Embodiment 47. The aqueous composition of Embodiment 41, wherein the preservative comprises one or more of sodium benzoate, benzyl alcohol, methyl paraben, propyl paraben, or any combination thereof.

Embodiment 48. The aqueous composition of Embodiment 41, wherein the cooling agent comprises menthol.

Embodiment 49. The aqueous composition of any of Embodiments 29-48, further comprising ethanol, isopropanol, or a combination thereof.

Embodiment 50. The aqueous composition of any of Embodiments 29-49, further comprising a propellant, the propellant comprising isobutane, propane, N-butane, or any combination thereof.

Embodiment 51. An aqueous composition comprising: lidocaine; phenylephrine HCl; citric acid; and malic acid, wherein the aqueous composition has a pH of 3.5 to 4.1.

Embodiment 52. The aqueous composition of Embodiment 51 comprising 2 to 5 wt. % lidocaine.

Embodiment 53. The aqueous composition of either Embodiment 51 or 52 comprising 0.25 wt. % phenylephrine HCl.

Embodiment 54. The aqueous composition of any of Embodiments 51-53 comprising 3.6 wt. % citric acid.

Embodiment 55. The aqueous composition of any of Embodiments 51-54 comprising 1 to 5 wt. % malic acid.

Embodiment 56. The aqueous composition of any of Embodiments 51-55 further comprising 10 to 20 wt. % glycerin.

Embodiment 57. The aqueous composition of any of Embodiments 51-56 further comprising 0.01 to 0.1 wt. % propyl gallate.

Embodiment 58. The aqueous composition of any of Embodiments 51-57 further comprising 0.001 to 0.08 wt. % menthol.

Embodiment 59. The aqueous composition of any of Embodiments 51-58 further comprising 5 to 10 wt. % ethanol.

Embodiment 60. A method of treating pain or inflammation in a mammalian subject comprising topically administering a therapeutically effective amount of the composition according to any of Embodiments 1-59 to a mammalian subject in need thereof.

Embodiment 61. A spray device comprising the composition according to any one of Embodiments 1-59.

Embodiment 62. An aqueous composition comprising:
lidocaine; and
at least two weak acids, each having at least one carboxylic acid group with a pKa of 2 to 5,
wherein the aqueous composition has a pH of 3.5 to 4.1.

Embodiment 63. The aqueous composition of claim 62, comprising 2 to 5 wt. % lidocaine.

Embodiment 64. The aqueous composition of claim 62 or claim 63, further comprising phenylephrine HCl.

Embodiment 65. The aqueous composition of claim 64, comprising 0.25 wt. % phenylephrine HCl.

Embodiment 66. The aqueous composition of any of claims 62-65, wherein each of the at least two weak acids are selected from citric acid, malic acid, acetic acid, tartaric acid, or ascorbic acid.

Embodiment 67. The aqueous composition of any of claims 62-65, wherein one of the at least two weak acids is citric acid.

Embodiment 68. The aqueous composition of claim 67, wherein the other of the at least two weak acids is malic acid, acetic acid, tartaric acid, or ascorbic acid.

Embodiment 69. The aqueous composition of any of claims 62-66, wherein one of the at least two weak acids is malic acid.

Embodiment 70. The aqueous composition of claim 69, wherein the other of the at least two weak acids is citric acid, acetic acid, tartaric acid, or ascorbic acid.

Embodiment 71. The aqueous composition of any of claims 62-65, wherein the at least two weak acids are citric acid and malic acid.

Embodiment 72. The aqueous composition of any of claims 62-71, wherein the at least two weak acids are present in a ratio of 5:3 to 1:3 based on wt. %.

Embodiment 73. The aqueous composition of claim 7 comprising 3.6 wt. % citric acid and 1 wt. % malic acid.

Embodiment 74. The aqueous composition of any of claims 62-73 further comprising one or more of an antioxidant, a chelating agent, an emollient, a humectant, a preservative, a cooling agent, and a propellant.

Embodiment 75. The aqueous composition of claim 74, wherein the antioxidant comprises propyl gallate, ascorbic acid, sodium metabisulfite, potassium metabisulfite, alpha tocopherol, or any combination thereof.

Embodiment 76. The aqueous composition of claim 74, wherein the antioxidant comprises 0.01 to 0.1 wt. % propyl gallate.

Embodiment 77. The aqueous composition of claim 74, wherein the chelating agent comprises disodium ethylenediaminetetraacetic acid (“EDTA”), edetic acid, or a combination of both.

Embodiment 78. The aqueous composition of claim 74, wherein the emollient comprises one or more of Aloe Barbadensis leaf juice, shea butter, vitamin E or pharmaceutically acceptable salts thereof, or any combination thereof.

Embodiment 79. The aqueous composition of claim 74, wherein the humectant comprises one or more of propylene glycol, glycerin, or any combination thereof.

Embodiment 80. The aqueous composition of claim 74, wherein the preservative comprises one or more of sodium benzoate, benzyl alcohol, methyl paraben, propyl paraben, or any combination thereof.

Embodiment 81. The aqueous composition of claim 74, wherein the cooling agent comprises menthol.

Embodiment 82. The aqueous composition of any of claims 62-81, further comprising ethanol, isopropanol, or a combination thereof.

Embodiment 83. The aqueous composition of any of claims 62-82, further comprising a propellant, the propellant comprising isobutane, propane, N-butane, or any combination thereof.

Embodiment 84. An aqueous composition comprising:
lidocaine;
phenylephrine HCl; and
citric acid;
malic acid,
wherein the aqueous composition has a pH of 3.5 to 4.1.

Embodiment 85. The aqueous composition of claim 84 comprising 2 to 5 wt. % lidocaine.

Embodiment 86. The aqueous composition of claim 84 or claim 85 comprising 0.25 wt. % phenylephrine HCl.

Embodiment 87. The aqueous composition any of claims 84-86 comprising 3.6 wt. % citric acid.

Embodiment 88. The aqueous composition of any of claims 84 to 87 comprising 1 to 5 wt. % malic acid.

Embodiment 89. The aqueous composition of any of claims 84-88 further comprising 10 to 20 wt. % glycerin.

Embodiment 90. The aqueous composition of any of claims 84-89 further comprising 0.01 to 0.1 wt. % propyl gallate.

Embodiment 91. The aqueous composition of any of claims 84-69 further comprising 0.001 to 0.08 wt. % menthol.

Embodiment 92. The aqueous composition of any of claims 84-91 further comprising 5 to 10 wt. % ethanol.

Embodiment 93. A method of treating pain or inflammation in a mammalian subject comprising topically administering a therapeutically effective amount of the composition according to any of claims 84-92 to a mammalian subject in need thereof.

Embodiment 94. A spray device comprising the composition according to any one of claims 84-92.

Embodiment 95. An aqueous composition comprising:
lidocaine;
phenylephrine HCl; and
at least two weak acids selected from a group consisting essentially of citric acid, malic acid, acetic acid, tartaric acid, and ascorbic acid,
wherein the aqueous composition has a pH of 3.5 to 4.1.

Embodiment 96. An aqueous composition comprising:
lidocaine;
phenylephrine HCl; and
citric acid in combination with at least one weak acid having at least one carboxylic acid group with a pKa of 2 to 5,
wherein the aqueous composition has a pH of 3.5 to 4.1.

Embodiment 97. The aqueous composition of claims 95 or 96, comprising 2 to 5 wt. % lidocaine.

Embodiment 98. The aqueous composition of any of claims 95-97 comprising 0.25 wt. % phenylephrine HCl.

### V. EXAMPLES

Some embodiments described herein are further illustrated in the following non-limiting examples.

### Example 1

### Low pH Solutions and Free Base Lidocaine

One of the technical challenges for phenylephrine HCl-containing compositions is that the pH of the composition needs to be acidic to maintain phenylephrine stability, such as having a pH of 4.5 or lower, or ideally 4.1 or lower. However, the monograph-required free base form of lidocaine is not soluble in aqueous solutions at these pH values, and precipitates out.

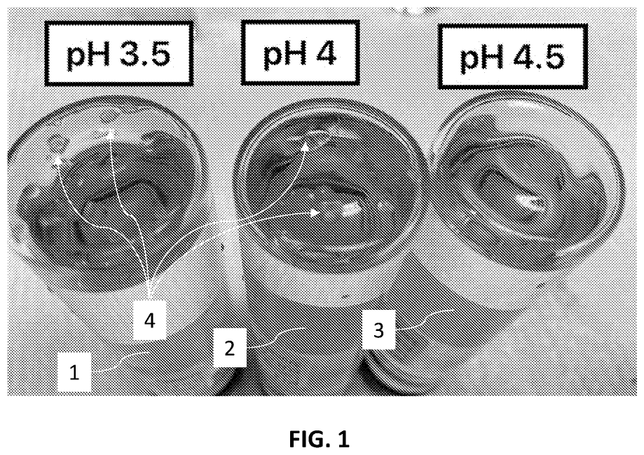

To confirm this precipitation problem, a composition was prepared having 5.0 wt. % lidocaine free base, 3.6 wt. % citric acid, 15 wt. % glycerin, 25 wt. % ethanol, and water. The pH was slowly adjusted between 4.5 to 3.5 by adding excess citric acid at 25° C. Precipitation of lidocaine was observed starting at pH=4.0 and increased in quantity the pH was further reduced to 3.5. FIG. 1 is a photograph of three vials of the above composition, with vial 1 being adjusted to pH=3.5, vial 2 being adjusted to pH=4.0, and vial 3 being adjusted to pH=4.5. As shown, no lidocaine free base crystallization was observed for vial 3 when the pH=4.5. However, as shown in vial 2, when the pH was adjusted to 4.0 by the addition of more citric acid, several lidocaine free base crystals 4 were observed to form. As the pH was further adjusted downward to 4.0 with the addition of more citric acid, more lidocaine free base crystals 4 were observed to form than those observed in vial 2. Accordingly, lidocaine free base has reduced solubility in pH solutions <4.5 when citric acid is used as a pH modifier.

While not intending to be bound by theory, while lidocaine free base was added to the aqueous solution, the addition of citric acid is believed to form the citrate salt form of lidocaine in situ. Thus, it is likely (but not analytically confirmed) that the crystals observed in vials 1 and 2 were actually lidocaine citrate rather than lidocaine free base.

### Example 2

### Crystallization Inhibitors and Free Base Lidocaine Crystallization in Low pH Solutions

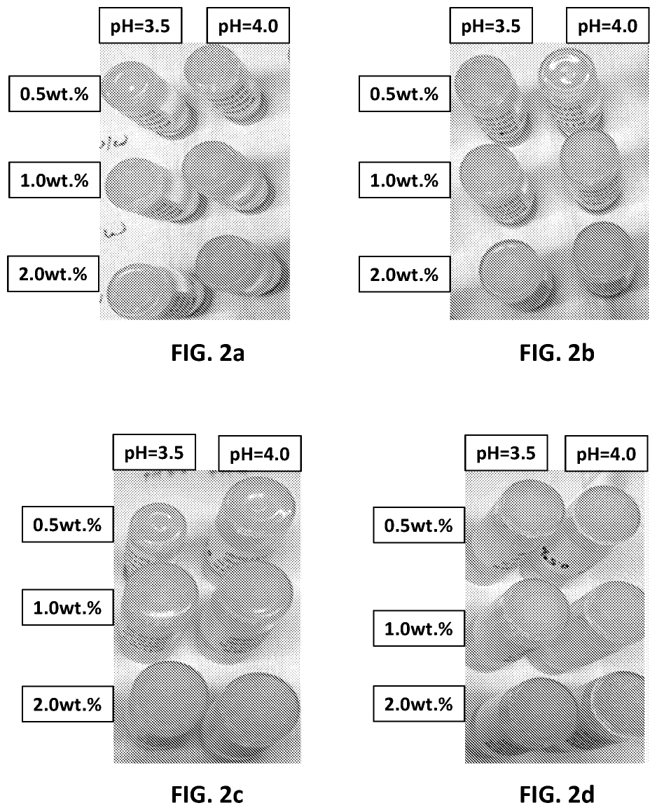

Different crystallization inhibitors were explored as a possible solution to lidocaine crystallization. To test the effectiveness of crystallization inhibitors, 0.5 wt. %, 1.0 wt. %, and 2.0 wt. % of Kollidon VA 64 (FIG. 2a), polyvinylpyrrolidone (PVP) K-90 (FIG. 2b), PVP K-25 (FIG. 2c), and PVP K-30 (FIG. 2d) were each added to duplicate, separate aqueous solutions having 5.0 wt. % lidocaine free base using the procedure and composition described in Example 1. The pH of one of each duplicate solution was then adjusted to have a pH=3.5 and the other duplicate solution to have a pH=4 by adding excess citric acid to give a total of 24 samples. FIGS. 2a-2d show the different samples as-prepared. For each of FIGS. 2a-2d, the left column is the lidocaine free base-containing solution adjusted to pH=3.5 using excess citric acid. The right column is the lidocaine free base-containing duplicate solution adjusted to pH=4.0 using excess citric acid. Additionally for each of FIGS. 2a-2d, the top row is for As shown in FIGS. 2a-2d, all of the samples displayed a thick layer of lidocaine crystallization on the sides of each vial. Consequently, it was determined that the use of crystallization inhibitors failed to prevent lidocaine crystallization in low pH solutions.

### Example 3

### Cosolvents and Lidocaine Citrate Crystallization in Low pH Solutions

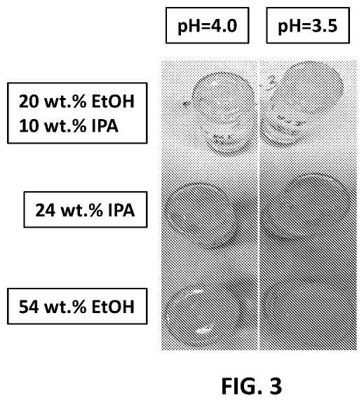

Various solutions having different combinations of solvents, such as aqueous alcohol solutions, were explored as a possible solution to the lidocaine crystallization problem. The use of alcohol as a co-solvent was predicted to increase lidocaine solubility in lower pH solutions. To test this theory, 5 wt. % lidocaine free base was dissolved into different duplicate vials having 1) a mixture of water and 24 wt. % isopropyl alcohol; 2) a mixture of water and 54 wt. % ethanol; and 3) a combination of water, 10 wt. % isopropyl alcohol and 20 wt. % ethanol. The pH of one of each duplicate solution was then adjusted to have a pH=3.5 and the other duplicate solution to have a pH=4 by adding excess citric acid to give a total of 6 samples. In FIG. 3, the top row is the combination of 10 wt. % isopropyl alcohol and 20 wt. % ethanol, the middle row is 24 wt. % isopropyl alcohol, and the bottom row is 54 wt. % ethanol. The left column of FIG. 3 shows the solutions adjusted to pH=4, and the right column shows the solutions adjusted to pH=3.5. As shown in FIG. 3, lidocaine citrate crystallized out of all samples as an opaque layer at both pH of 4.0 and 3.5 for all the different solvents. Thus, while the use of aqueous alcohol solutions can impart desirable physical characteristics of the composition (such as improved drying time) in some embodiments, the presence of the alcohol did not prevent lidocaine free base crystallization in low pH solutions.

### Example 4

### Single Weak Acid Lidocaine Salts and Crystallization in Low pH Solutions

In situ generation of different salt forms of lidocaine were investigated for increasing lidocaine solubility in low pH solutions with marginal improvements being observed. For instance, lidocaine citrate was observed to increase solubility of lidocaine in mildly acidic solutions (pH=>4.6), lidocaine citrate was also observed to crystallize out in lower pH solutions (pH=<4.5), as seen for instance in Example 1. For example, a 1:1 molar ratio of lidocaine free base and citric acid aqueous solution was prepared to form the lidocaine citrate salt in situ. The pH of the resulting solution was 4.6. However, since PE degrades faster in solutions having a pH greater than 4.0, the pH was attempted to be reduced to 3.5-4.0 by adding excess citric acid. The lidocaine citrate was observed to precipitate out at lower temperatures (4° C.) as the pH was reduced from 4.6 to 4.0 and lower. While not intending to be bound by theory, it is believed the precipitation effects are due to the common ion effect, where, as the excess citric acid precipitates out as the pH is lowered, the citrate salt of lidocaine citrate co-crystallizes with the precipitating citric acid and pulls the lidocaine out of the solution in the process.

In-situ generation of lidocaine malate was achieved by using a 1:1 molar ratio of lidocaine free base and malic acid in water. Excess malic acid was then added to reduce the pH to 3.6. No lidocaine malate crystallization was observed at this pH after several weeks of storing the sample at 4° C. Thus, the single salt form of lidocaine malate was observed to be stable in aqueous solutions having lower pH. However, to achieve this pH range, 5 wt. % of malic acid was required. As previously discussed, at amounts of 5 wt. % or higher, malic acid has been observed to be an irritant to a healthy skin, and likely could be irritant at amounts less than 5 wt. % on affected or damaged skin. Consequently, while lidocaine malate salt appears to be more stable than the citrate salt, the malate salt form of lidocaine is not a practical approach for compositions that can be applied to irritated or damaged skin (i.e. hemorrhoids, cuts, and the like).

### Example 5

Two Weak Acid Combinations with Lidocaine and Crystallization in Low pH Solutions

It was discovered that the use of at least two weak acids in combination increase solubility of lidocaine in low pH solutions of pH=<4.5, such as 3.5-4.1, and prevent lidocaine crystallization. Specifically, the use of at least two weak acids having at least one carboxylic acid group with a pKa of 2 to 5 increases solubility of lidocaine in low pH solutions. While not intending to be bound by theory, it is believed that the use of two weak acids alleviates the common ion effect and prevents lidocaine citrate from precipitating out of solution at lower pH of 3.5-4.1.

A 1:1 molar ratio of lidocaine free base and citric acid were added to an aqueous solution comprising 25 wt. % ethanol, 15 wt. % glycerin, and 2.5 wt. % propylene glycol to form the lidocaine citrate salt in situ, with the pH of the resulting solution being approximately 4.6. However, rather than using excess citric acid to reduce the pH, as done in Example 4, different acidifying agents were added to in an amount of 2 wt. %. The pH was then measured, and stability was tested by keeping the resulting solutions at 4° C. for 3 months. Table 1 summarizes the different acidifying agents used to adjust the pH, any precipitation observed, and any additional observations.

TABLE 1
Acidifying Agents in Combination with Lidocaine Citrate

| | | Time to | |
|---|---|---|---|
| | | Lidocaine | |
| Acidifying | | Citrate | |
| agent | pH | Precipitation | Observation |
| Maleic acid | 3.32 | Not observed | Oxidized |
| L-Tartaric acid | 3.71 | Not observed | Clear |
| Fumaric acid | 3.72 | Not observed | Oxidized |
| Malic Acid | 3.93 | Not observed | Clear |
| Lactic acid | 4.05 | Not observed | Clear |
| Succinic acid | 4.23 | Not observed | Clear |
| Ascorbic acid | 4.02* | Not observed | Oxidized |
| Adipic Acid | 4.17* | 2 weeks | Clear |
| Acetic Acid | 4.00 | Not observed | Clear |

*5% acidifying agent was added because 2% did not reduce the pH significantly

The target pH of the resulting solutions was <4.0 for purposed of maximizing PE stability, which most of the different acidifying agents achieved at 2 wt. % addition. However, there were exceptions to this goal. Ascorbic acid and adipic acid did not sufficiently reduce the pH when present at 2 wt. %, so their amounts were increased to 5 wt. %. This increased quantity is unfortunately undesirable since it increases the cost and complexity of composition.

As summarized in Table 1, addition of a second acidifying agent (weak acids) prevented precipitation of lidocaine citrate across a pH range of 4.2 to 3.3, with the exception of adipic acid which did not prevent precipitation. It was noted that while maleic acid (2%), fumaric acid (2%), and ascorbic acid (5%) prevented lidocaine citrate precipitation, these samples were observed to change from clear solutions to pale yellow solutions (or orange for ascorbic acid) under ambient conditions in less than 2 months' time indicating oxidation of the acidifying agents.

In summary, when the pH of a lidocaine citrate-containing solution is adjusted using a second acidifying agent (weak acid) rather than just using an excess of citric acid, precipitation is lidocaine is reduced or eliminated.

1. An aqueous composition comprising:
lidocaine; and
at least two weak acids, each having at least one carboxylic acid group with a pKa of 2 to 5,
wherein the aqueous composition has a pH of 3.5 to 4.1.

2. The aqueous composition of claim 1, comprising 2 to 5 wt. % lidocaine.

3. The aqueous composition of claim 1, further comprising phenylephrine HCl.

4. The aqueous composition of claim 3, comprising 0.25 wt. % phenylephrine HCl.

5. The aqueous composition of claim 1, wherein each of the at least two weak acids is citric acid, malic acid, acetic acid, tartaric acid, or ascorbic acid.

6. The aqueous composition of claim 1, wherein one of the at least two weak acids is citric acid.

7. The aqueous composition of claim 6, wherein the other of the at least two weak acids is malic acid, acetic acid, tartaric acid, or ascorbic acid.

8. The aqueous composition of claim 1, wherein one of the at least two weak acids is malic acid.

9. The aqueous composition of claim 8, wherein the other of the at least two weak acids is citric acid, acetic acid, tartaric acid, or ascorbic acid.

10. The aqueous composition of claim 1, wherein the at least two weak acids are citric acid and malic acid.

11. The aqueous composition of claim 1, wherein the at least two weak acids are present in a ratio of 5:3 to 1:3 based on wt. %.

12. The aqueous composition of claim 10 comprising 3.6 wt. % citric acid and 1 wt. % malic acid.

13. The aqueous composition of claim 1, further comprising one or more of an antioxidant, a chelating agent, an emollient, a humectant, a preservative, a cooling agent, and a propellant.

14. The aqueous composition of claim 13, wherein the antioxidant comprises propyl gallate, ascorbic acid, sodium metabisulfite, potassium metabisulfite, alpha tocopherol, or any combination thereof.

15. The aqueous composition of claim 13, wherein the antioxidant comprises 0.01 to 0.1 wt. % propyl gallate.

16. The aqueous composition of claim 13, wherein the chelating agent comprises disodium ethylenediaminetetraacetic acid (“EDTA”), edetic acid, or a combination of both.

17. The aqueous composition of claim 13, wherein the emollient comprises one or more of Aloe Barbadensis leaf juice, shea butter, vitamin E or pharmaceutically acceptable salts thereof, or any combination thereof.

18. The aqueous composition of claim 13, wherein the humectant comprises one or more of propylene glycol, glycerin, or any combination thereof.

19. The aqueous composition of claim 13, wherein the preservative comprises one or more of sodium benzoate, benzyl alcohol, methyl paraben, propyl paraben, or any combination thereof.

20. The aqueous composition of claim 13, wherein the cooling agent comprises menthol.

21. The aqueous composition of claim 1, further comprising ethanol, isopropanol, or a combination thereof.

22. The aqueous composition of claim 1, further comprising a propellant, the propellant comprising isobutane, propane, N-butane, or any combination thereof.

23. An aqueous composition comprising:
lidocaine;
phenylephrine HCl;
citric acid; and
malic acid,
wherein the aqueous composition has a pH of 3.5 to 4.1.

24. The aqueous composition of claim 23 comprising 2 to 5 wt. % lidocaine.

25. The aqueous composition of claim 23 comprising 0.25 wt. % phenylephrine HCl.

26. The aqueous composition of claim 23 comprising 3.6 wt. % citric acid.

27. The aqueous composition of claim 23 comprising 1 to 5 wt. % malic acid.

28. The aqueous composition of claim 23, further comprising 10 to 20 wt. % glycerin.

29. The aqueous composition of claim 23, further comprising 0.01 to 0.1 wt. % propyl gallate.

30. The aqueous composition of claim 23, further comprising 0.001 to 0.08 wt. % menthol.

31. The aqueous composition of claim 23, further comprising 5 to 10 wt. % ethanol.

32. A method of treating pain or inflammation in a mammalian subject comprising topically administering a therapeutically effective amount of the composition according to claim 1 to a mammalian subject in need thereof.

33. A spray device comprising the composition according to claim 1.

34. An aqueous composition comprising:
lidocaine;
phenylephrine HCl; and
at least two weak acids selected from a group consisting essentially of citric acid, malic acid, acetic acid, tartaric acid, and ascorbic acid,
wherein the aqueous composition has a pH of 3.5 to 4.1.

35. An aqueous composition comprising:
lidocaine;
phenylephrine HCl; and
citric acid in combination with at least one weak acid having at least one carboxylic acid group with a pKa of 2 to 5,
wherein the aqueous composition has a pH of 3.5 to 4.1.

36. The aqueous composition of claim 34, comprising 2 to 5 wt. % lidocaine.

37. The aqueous composition of claim 34, comprising 0.25 wt. % phenylephrine HCl.
